(19) United States
(12) Patent Application Publication
(10) Pub. No.: US 2026/0325023 A1
(43) Pub. Date: Oct. 22, 2026

(54) Method and System for Enhanced Photoplethysmography Using Controlled Finger Curling Over a Light Sensor

(71) Applicant: Edward Christo Kezeli, Rescue, CA (US)

(72) Inventor: Edward Christo Kezeli, Rescue, CA (US)

(21) Appl. No.: 19/565,451

(22) Filed: Mar. 12, 2026

Related U.S. Application Data
(60) Provisional application No. 63/777,565, filed on Mar. 25, 2025.

(51) Int. Cl. A61B 5/1455; A61B 5/00; A61B 5/021

(52) U.S. Cl. CPC A61B 5/14552; A61B 5/02108; A61B 5/6898; A61B 5/7257; A61B 5/742; A61B 5/7455

(57) ABSTRACT

A method and system to enhance photoplethysmography (PPG) signals using a portable electronic device's light sensor and light source. Rather than placing the finger flat on the sensor, the user curls the finger pad by bending the joints to an angle of 5° to 60°. This controlled curling action increases the amount of finger tissue in the light path, resulting in a stronger and clearer PPG signal with approximately 1.25 to 2 times higher amplitude compared to the conventional flat position. The enhanced signal provides more accurate and reliable monitoring of physiological parameters such as heart rate, pulse variability, blood oxygen saturation, and blood pressure using only standard consumer devices.

### CROSS-REFERENCE TO RELATED APPLICATIONS

This application claims the benefit of U.S. Provisional Patent Application No. 63/777,565 filed on Mar. 25, 2025.

### BACKGROUND OF THE INVENTION

This disclosure relates to optical sensing, specifically to improving PPG signal acquisition using a light sensor in a portable electronic device. It focuses on enhancing signal amplitude and fidelity through controlled finger positioning to optimize light interaction with vascular tissue across visible and infrared wavelengths.

Photoplethysmography (PPG) is a widely adopted non-invasive optical technique that measures blood volume changes in microvascular tissue beds, enabling the monitoring of physiological parameters such as heart rate, pulse variability, and blood oxygen saturation. This occurs in devices including smartphones, wearables, and medical monitors, where a finger is typically placed flat against a light sensor, such as a camera lens or photodiode, and illuminated by a light source, often an LED, to capture pulsatile signals.

Conventional static placement at 0° (flat) can yield a baseline PPG signal with suboptimal amplitude and fidelity due to thick epidermis, anatomical variations from age or physiology, or limited finger tissue coverage over the sensor's active area, reducing exposure to internal vasculature. These limitations hinder light penetration into deeper vascular tissues and reduce the pulsatile response of blood vessels, impairing accurate detection of arterial fluctuations. Prior art relies on signal processing to enhance the PPG waveform rather than optimizing physical interaction, leaving a need for a method using standard hardware to enhance PPG signals dynamically via user action.

### SUMMARY OF THE INVENTION

Embodiments provide a method and system for enhancing PPG signals by curling a finger over a light sensor in a portable device, using standard hardware with optional feedback. This approach adjusts finger positioning to utilize physiological properties and optimize light-tissue interaction, thereby improving the accuracy and reliability of physiological parameter measurements.

Curling the finger joints from 5-60° increases signal amplitude and enhances fidelity. The system comprises a light sensor, light source, and processor, with optional dynamic sensor adjustments and user feedback to guide curling.

### BRIEF DESCRIPTION OF THE DRAWINGS

FIG. 1 is a schematic view of an exemplary portable electronic device configured for PPG signal detection.

FIG. 2a (flat) is a side view illustrating a finger pad positioned flat (0°) on a light sensor (102) with a light sensor path (106) indicating the field of view, and an adjacent LED light source (104).

FIG. 2b (curled) is a side view depicting a finger curled over a light sensor (102) with a light sensor path (106) indicating the field of view, showing angles such as 22° at the proximal interphalangeal joint and an additional 18° at the distal interphalangeal joint, shifting the contact 1-15 mm toward the fingertip.

FIG. 3 is a side view of a curled finger over a light sensor (102) with a light sensor path (106) indicating the field of view, and an inset (118) showing a flat position (118a) and a curled position (118b) with a 1-15% increase in effective finger tissue volume in the optical path (as determined by the increased cross-sectional area) exposed to the light sensor compared to a flat position.

FIG. 4 is a set of graphs showing PPG signals from a flat finger at 0° (112), a curled finger at 5-60° (114), and a comparison graph (116) illustrating a 1.25-2 times amplitude increase from the flat to the curled finger.

### DETAILED DESCRIPTION OF THE INVENTION

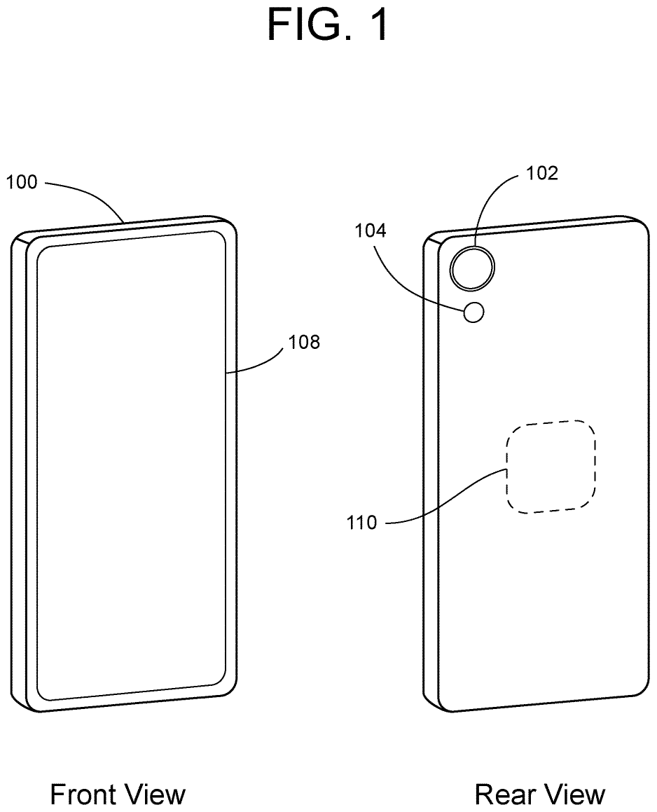

This disclosure provides a method and system for enhancing PPG signal acquisition via controlled finger curling over a light sensor. FIG. 1 shows a portable electronic device (100), such as a smartphone, wearable, or tablet, with a light sensor (102), light source (104), display (108), and processor (110).

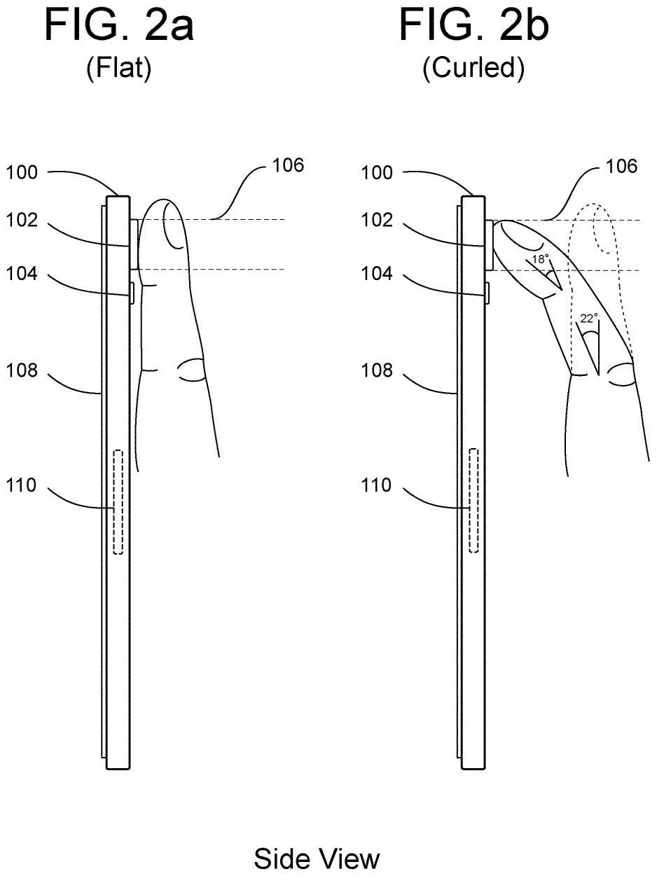

The method starts by positioning a finger pad-the fleshy ventral portion of the distal phalanx, encompassing the area typically used for fingerprinting-on the light sensor (102) at 0° (flat), per FIG. 2 a, establishing a baseline, then curling the finger by flexing the proximal interphalangeal joint, distal interphalangeal joint, or both (optionally with the metacarpophalangeal joint) to 5-60°. In some embodiments, curling may include pressing and sliding the finger downward to enhance contact and signal capture, shifting the contact 1-15 mm toward the fingertip, as in FIG. 2 b.

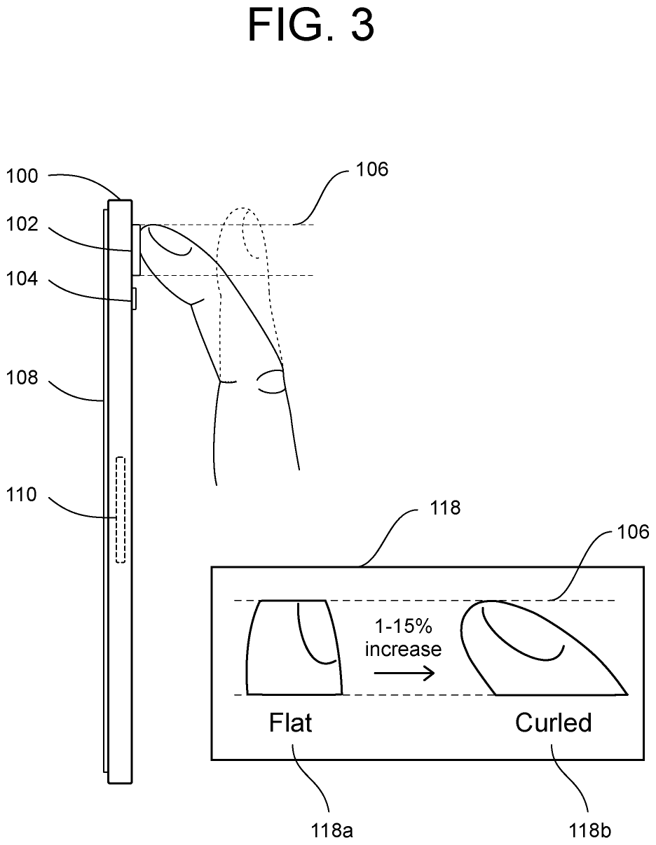

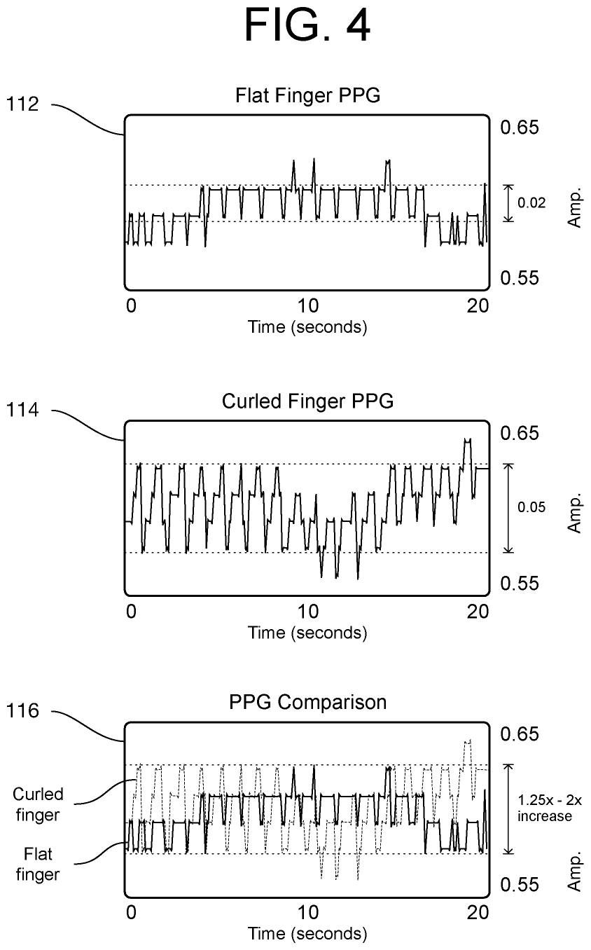

Curling enhances signal amplitude and fidelity by resulting in optimized light-tissue interaction, increased vascular exposure in the optical path, and improved mechanical contact/pressure, with a 1-15% greater effective finger tissue volume in the optical path (as determined by the increased cross-sectional area of tissue intersecting the sensor's (102) field of view (106)), shown in FIG. 3. This increased volume of finger tissue in the optical path provides greater interaction with subsurface vascular structures, resulting in enhanced clarity and consistency of the arterial pulse waveform, as illustrated in FIG. 4, which presents a set of graphs of PPG signals demonstrating the amplitude improvement with curling.

An application on processor (110) activates light source (104) to emit visible (400-700 nm) or infrared (700-1100 nm) light, illuminating the tissue continuously or in pulses synced with sensor detection, and instructs the user via display (108) to curl the finger. The light sensor (102) captures signals at a suitable rate (e.g. 30-120 fps for cameras) for 10 seconds to 60 seconds for typical measurements, or extends indefinitely (e.g., up to 30 minutes or beyond). Processor (110) applies peak detection, Fast Fourier Transform, or artificial intelligence models to extract parameters like heart rate, pulse variability, blood oxygen saturation, or blood pressure.

To optimize capture, sensor settings-e.g., exposure and white balance-adjust dynamically to ambient light conditions, skin tone, or curl angle. Feedback, which may be visual (e.g., via display graphics) or haptic (e.g., via vibrations of 50-500 milliseconds), guides the user to maintain a 5-60° curl angle. If curling deviates beyond 60°, feedback adjusts positioning to return to the 5-60° range.

The method accommodates variations. A curl of at least 5° at any joint begins to increase amplitude, with a curl of 10° or more reliably achieving at least a 1.25× increase, as shown in FIG. 4. Alternative fingers (middle, ring, pinky, thumb) function similarly. Curling accommodates thicker skin (>1 mm epidermis) and physiological differences, including variations in vasculature, and weak signals from cold hands, enhancing signal capture when the sensor is covered. Pressing the finger pad may augment amplitude (1.1×-1.5×), though curling remains primary. Testing showed a ≥1.25× amplitude increase even with cold hands, where baseline signals are typically weaker due to reduced peripheral perfusion, suggesting that curling provides mechanical advantages (such as improved contact and tissue positioning) that overcome these limitations.

1. A method for acquiring an enhanced PPG signal using a portable electronic device, comprising:
a) illuminating a finger with a light source operable to emit light in a spectrum including visible and infrared wavelengths;
b) positioning a finger pad, defined as the fleshy ventral portion of a finger's distal phalanx, encompassing the area typically used for fingerprinting, onto a light sensor of a portable electronic device;
c) curling the finger from a flat orientation (0°) by flexing at least one of a proximal interphalangeal joint or a distal interphalangeal joint to an angle of 5-60°;
d) detecting, via the light sensor, light modulated by blood volume changes within the curled finger; and
e) processing the detected light with a processor to generate a PPG signal, wherein curling increases PPG waveform amplitude by approximately 1.25-2 times and enhances signal fidelity, defined as clarity and consistency of arterial pulse waveform, relative to a static flat finger placement at 0°, through improved light penetration, vascular exposure, and optimized tissue positioning and contact, with a 1-15% greater effective finger tissue volume in the light path over the sensor (as determined by the increased cross-sectional area of tissue intersecting the sensor's field of view).

2. The method of claim 1, wherein the finger pad is positioned on the sensor's active area to cover the sensor.

3. The method of claim 1, wherein curling by at least 10° at any single joint increases amplitude by at least 1.25 times relative to the flat placement.

4. The method of claim 1, wherein curling comprises flexing at least one interphalangeal joint, optionally with a metacarpophalangeal joint.

5. The method of claim 1, wherein the finger is an index finger, middle finger, ring finger, pinky finger, or thumb.

6. The method of claim 1, wherein the device is a smartphone, wearable, or tablet, the light source is an LED operable to emit light across 400-700 nanometers for visible light or 700-1100 nanometers for infrared light, and the light sensor is a camera lens or photodiode.

7. The method of claim 1, wherein detection occurs over 10 seconds to 60 seconds for typical physiological measurements, or extends indefinitely based on user requirements or the physiological parameter being monitored.

8. The method of claim 1, further comprising dynamically adjusting sensor settings, including exposure or white balance, responsive to curl angle, skin pigmentation, or ambient light conditions.

9. The method of claim 1, further comprising providing feedback, which may be visual or haptic, via a display graphic adjusting from 5-60°, or haptic feedback via vibration, guiding the curl angle.

10. The method of claim 1, wherein processing uses peak detection, Fast Fourier Transform, or artificial intelligence models to extract heart rate, pulse variability, blood oxygen saturation, or blood pressure.

11. The method of claim 1, further comprising pressing and sliding the finger downward to enhance contact and signal capture.

12. The method of claim 1, wherein curling improves signal acquisition from cold hands, including cases where baseline PPG signals are attenuated due to peripheral vasoconstriction.

13. A system for acquiring an enhanced PPG signal, comprising:
a) a light sensor configured to capture light reflected from a finger, integrated into a portable electronic device, such as a smartphone, wearable or tablet;
b) a light source configured to illuminate a finger curled from a flat orientation by flexing at least one of a proximal interphalangeal joint or a distal interphalangeal joint to an angle of 5-60° relative to the flat orientation, operable to emit light across 400-700 nanometers for visible light or 700-1100 nanometers for infrared light; and
c) a processor configured to:
i) detect light modulated by blood volume changes within the curled finger; and
ii) process the detected light to generate a PPG signal, wherein curling increases PPG waveform amplitude by approximately 1.25-2 times and enhances signal fidelity relative to a flat placement at 0° through improved light penetration, vascular exposure, and optimized tissue positioning and contact, with a 1-15% greater effective finger tissue volume in the light path over the sensor (as determined by the increased cross-sectional area of tissue intersecting the sensor's field of view).

14. The system of claim 13, wherein the processor is configured to adjust sensor settings to optimize signal acquisition under varying conditions.
